(19) United States
(12) Patent Application Publication
(10) Pub. No.: US 2025/0228950 A1
(43) Pub. Date: Jul. 17, 2025

(54) Prodrugs Activated by Reduction in the Cytosol

(71) Applicant: YALE UNIVERSITY, New Haven, CT (US)

(72) Inventors: John Deacon, New Haven, CT (US); Kung-Pern Wang, New Haven, CT (US); Venkatareddy Sabbasani, New Haven, CT (US)

(21) Appl. No.: 18/930,103

(22) Filed: Oct. 29, 2024

Related U.S. Application Data
(63) Continuation-in-part of application No. 16/769,523, filed on Jun. 3, 2020, now abandoned.
(60) Provisional application No. 62/595,319, filed on Dec. 6, 2017.

(51) Int. Cl. A61K 47/54

(52) U.S. Cl. CPC A61K 47/54; A61K 47/548

(57) ABSTRACT

The present invention provides prodrug modifications for chemotherapeutic drugs that allow activation in the cytosol.

### CROSS REFERENCE TO RELATED APPLICATIONS

The present application claims priority under 35 U.S.C. § 119(e) to U.S. Provisional Patent Application No. 62/595,319, filed Dec. 6, 2017, which is incorporated herein by reference in its entirety.

### STATEMENT REGARDING FEDERALLY SPONSORED RESEARCH OR DEVELOPMENT

This invention was made with government support under CA106359, GM073857, and P30CA016359 awarded by National Institutes of Health. The government has certain rights in the invention.

### BACKGROUND OF THE INVENTION

Prodrug approaches to cancer chemotherapy generally share a common limitation: they are activated in the body in a given organ (such as the liver), then circulate as active drugs before they can permeate tumors and exert antitumor effects. This inefficient process leads to squandered activity and myelotoxic side effects, among others, which limit the therapeutic potential of these treatments.

There is thus a need in the art for novel chemotherapies, which allow for the active compound to be unmasked and/or released within the tumor itself, with greater specificity and limited side effects. The present invention addresses this need.

### BRIEF SUMMARY OF THE INVENTION

In one aspect, the invention provides a compound of formula (1): R-Linker-Drug (1), which is selected from the group consisting of:

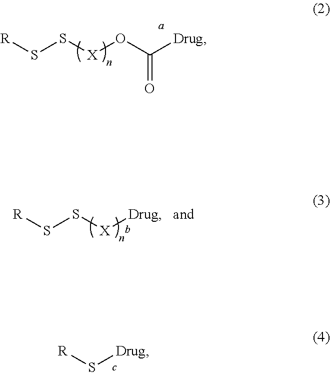

wherein: Drug is a chemotherapy drug; each instance of R is independently selected from the group consisting of a biomarker targeting moiety, a tumor targeting moiety, a DNA targeting moiety, C₁-C₆ alkyl, and aryl; each occurrence of n is independently an integer ranging from 1 to 4; each occurrence of X is independently selected from the group consisting of CH₂, CH(alkyl), and C(alkyl)₂; bond a is formed between the carbon and a substituent on Drug, wherein the substituent is selected from the group consisting of primary amine, secondary amine, and hydroxyl; bond b is formed between the carbon and a substituent on Drug, wherein the substituent is selected from the group consisting of hydroxyl, carboxyl, amide, and phosphoramide; and bond c is formed between the carbon and a substituent on Drug, wherein the substituent is a sulfur atom; or a salt, solvate, enantiomer, diastereomer, geometric isomer, or tautomer thereof.

In various embodiments, the Drug is 3-methyl-(triazen-1-yl)imidazole-4-carboxamide (MTIC), or a salt or solvate thereof.

In various embodiments, the compound of formula (1) is:

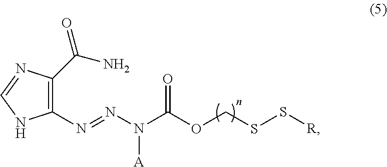

wherein A is selected from the group consisting of alkyl, haloalkyl, benzyl, halobenzyl, methyl, 2-chloroethyl, and ethyl methanesulfonate, or a salt, solvate, enantiomer, diastereomer, geometric isomer or tautomer thereof.

In various embodiments, the Drug is a Pyrrolobenzodiazepine.

In various embodiments, the compound of formula (1) is:

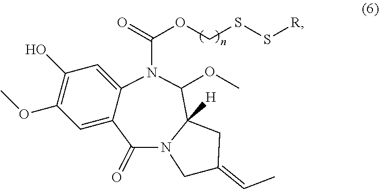

or a salt, solvate, enantiomer, diastereomer, geometric isomer or tautomer thereof.

In various embodiments, the Drug is a nitrogen mustard.

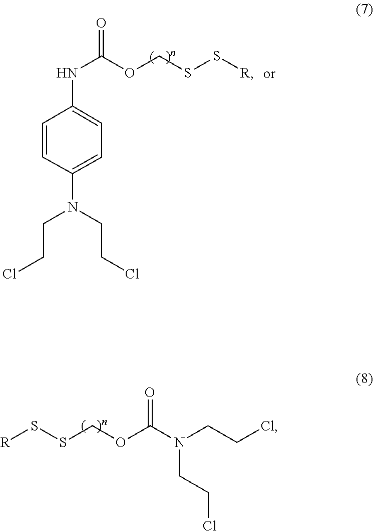

In various embodiments, the compound of formula (1) is:

or a salt, solvate, enantiomer, diastereomer, geometric isomer or tautomer thereof.

In various embodiments, the Drug is 12-hydroxyellipticine or 13-hydroxyellipticine.

In various embodiments, the compound of formula (1) is:

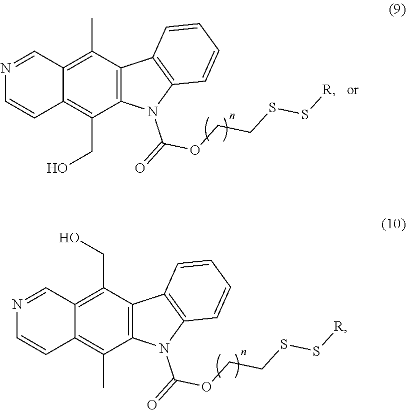

or a salt, solvate, enantiomer, diastereomer, geometric isomer or tautomer thereof.

In various embodiments, the Drug is a nitrosocarbamate.

In various embodiments, the compound of formula (1) is:

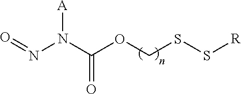

wherein A is selected from the group consisting of alkyl, haloalkyl, benzyl, halobenzyl, methyl, 2-chloroethyl, and ethyl methanesulfonate, or a salt, solvate, enantiomer, diastereomer, geometric isomer or tautomer thereof.

In various embodiments, the compound of formula (1) is:

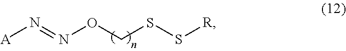

wherein A is selected from the group consisting of alkyl, haloalkyl, benzyl, halobenzyl, methyl, 2-chloroethyl, and ethyl methanesulfonate,

In various embodiments, the Drug is 5-(3-hydroxymethyl-3-methyl-1-triazeno)imidazole-4-carboxamide (HMMTIC).

In various embodiments, the compound of formula (1) is:

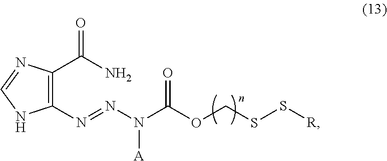

wherein A is selected from the group consisting of alkyl, haloalkyl, benzyl, halobenzyl, methyl, 2-chloroethyl, and ethyl methanesulfonate, or a salt, solvate, enantiomer, diastereomer, geometric isomer or tautomer thereof.

In various embodiments, Drug is a phosphoramide mustard.

In various embodiments, the compound of formula (1) is:

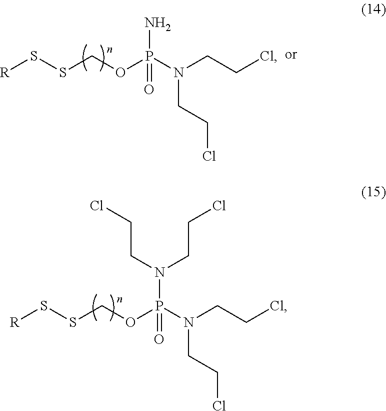

or a salt, solvate, enantiomer, diastereomer, geometric isomer or tautomer thereof.

In various embodiments, the Drug is o-thioquinone methide.

In various embodiments, the compound of formula (1) is:

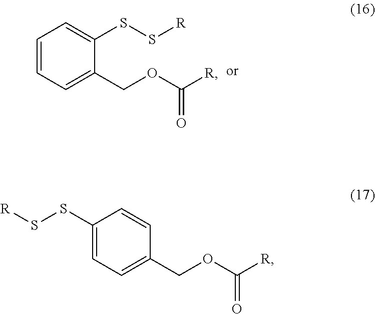

or a salt, solvate, enantiomer, diastereoisomer, geometric isomer or tautomer thereof.

In another aspect, the invention provides a compound of formula (18):

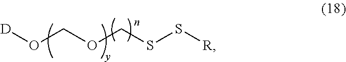

wherein: D is a DNA binding or nuclear localizing moiety; R is selected from the group consisting of a biomarker targeting moiety, a tumor targeting moiety, a DNA targeting moiety, C₁-C₆ alkyl, and aryl; each occurrence of n is independently an integer ranging from 1 to 4; y is 0-20; or a salt, solvate, enantiomer, diastereoisomer, geometric isomer or tautomer thereof.

In various embodiments, wherein y is 2.

In various embodiments, R is a weakly acidic group having a pKa between about 4.5 and about 7.5.

In various embodiments, R is selected from the group consisting of:

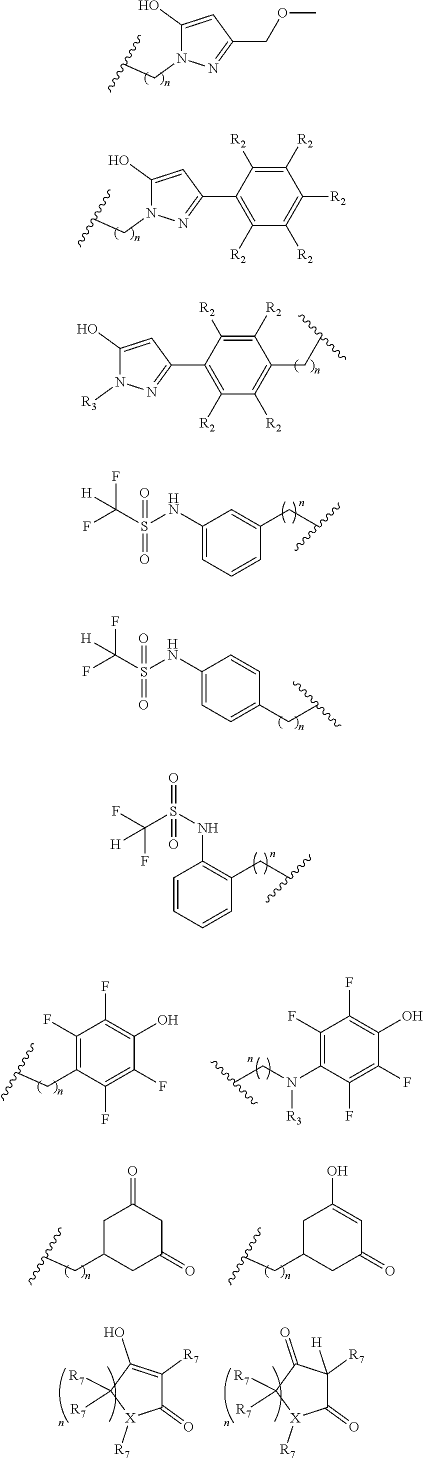

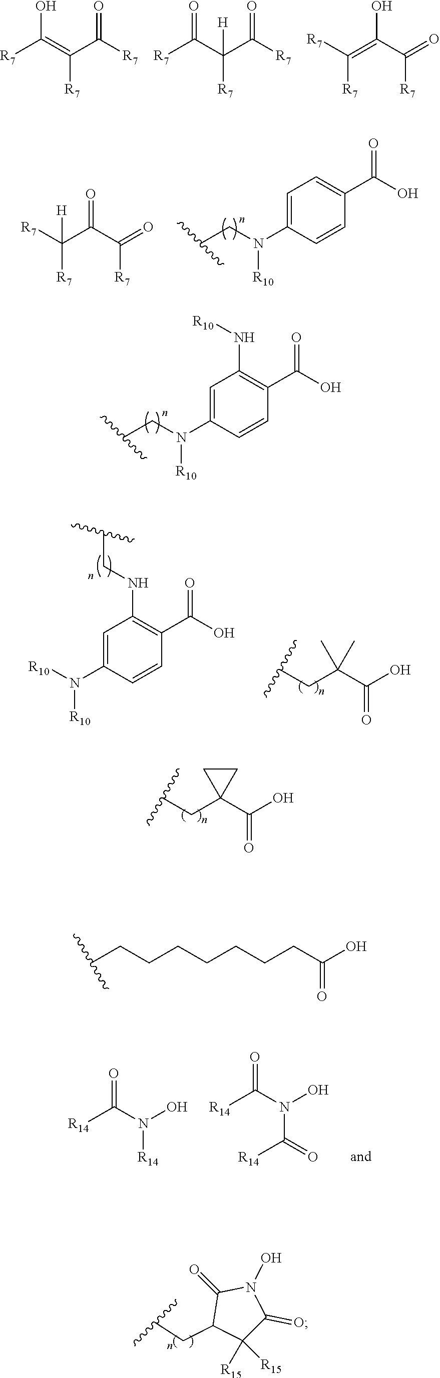

wherein each instance of R₂ is independently selected from the group consisting of H, F, Cl, hydroxy, methoxy, —NH₂, —NH-alkyl, —N(alkyl)₂, and alkyl; R₃ is selected from the group consisting of H, methyl, ethyl, alkyl, phenyl, benzyl, haloaryl, —CH₂—O—CH₃, and —CH₂—CH₂—OH; each instance of R₇ is independently selected from the group consisting of H, alkyl, phenyl, benzyl, an electron donating group, or a covalent bond to linker or drug; X is CH, C-alkyl, or N; at least one R₇ group comprises a covalent bond to Linker or Drug either directly or by displacing a hydrogen on alkyl, phenyl, benzyl or an electron donating group; each instance of R₁₀ is independently selected from the group consisting of H, alkyl, or an electron donating group; each instance of R₁₄ is independently an electron withdrawing group, an electron donating group or a covalent bond to Linker or Drug; at least one R₁₄ group comprises a covalent bond to Linker or Drug either directly or by displacing a hydrogen on an electron withdrawing or electron donating group; each instance of R₁₅ is independently selected from the group consisting of: H, an electron withdrawing group or an electron donating group; and n is an integer ranging from 0 to 4.

In various embodiments, the compound is selected from the group consisting of:

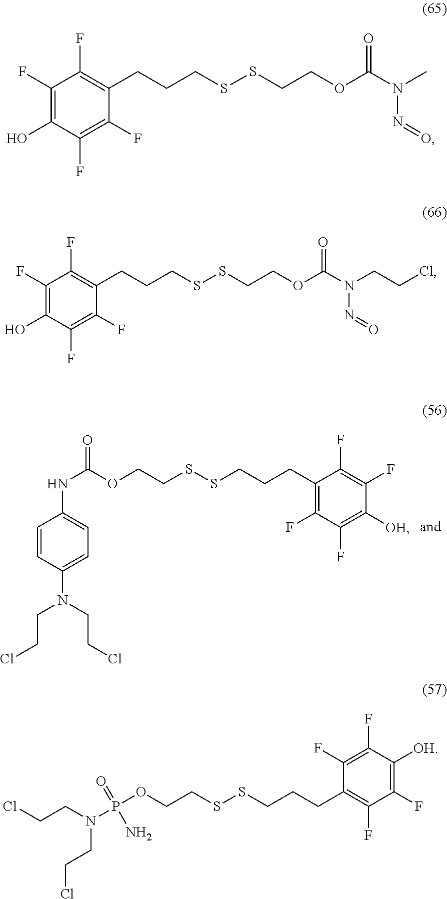 [SMILES: CN(N=O)C(=O)OCCSSCCCc1c(F)c(F)c(O)c(F)c1F ; O=NN(CCCl)C(=O)OCCSSCCCc1c(F)c(F)c(O)c(F)c1F ; O=C(Nc1ccc(N(CCCl)CCCl)cc1)OCCSSCCCc1c(F)c(F)c(O)c(F)c1F ; NP(=O)(OCCSSCCCc1c(F)c(F)c(O)c(F)c1F)N(CCCl)CCCl]

In various embodiments, the invention provides a pharmaceutical composition comprising a compound of the invention and a pharmaceutically acceptable carrier.

In various embodiments, pharmaceutical composition further comprises at least one additional therapeutic drug.

In various embodiments, the pharmaceutical composition is formulated for nasal, inhalational, topical, oral, buccal, rectal, pleural, peritoneal, vaginal, intramuscular, subcutaneous, transdermal, epidural, intratracheal, otic, intraocular, intrathecal, or intravenous administration.

In various embodiments, the invention provides a method for treating a cancer in a subject in need thereof, the method comprising administering to the subject a therapeutically effective amount of the compound or the pharmaceutical composition as described herein.

In various embodiments, the compound undergoes reduction to an active form in the cytosol of a cancer cell in the subject.

In various embodiments, the cancer is at least one selected from the group consisting of melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-Hodgkins lymphoma, hepatocellular carcinoma, bladder cancer, and lung cancer.

In various embodiments, the method further comprises procuring the compound or the pharmaceutical composition.

In various embodiments, the method further comprises administering to the subject an additional cancer treatment.

In various embodiments, the additional cancer treatment is radiation, surgical excision, immunotherapy, and antiproliferative chemotherapy.

In various embodiments, the invention provides a prepackaged pharmaceutical composition comprising the compound or the pharmaceutical composition and an instructional material for use thereof, wherein the instructional material comprises instructions for preventing or treating cancer in a subject.

### BRIEF DESCRIPTION OF THE DRAWINGS

The following detailed description of specific embodiments of the invention will be better understood when read in conjunction with the appended drawings. For the purpose of illustrating the invention, certain specific embodiments are shown in the drawings. It should be understood, however, that the invention is not limited to the precise arrangements and instrumentalities of the embodiments shown in the drawings.

FIG. 1 is a graph depicting kinetics of MTIC release following reduction of the prodrug YU252215.

FIG. 2 is a graph depicting an A375 cell growth inhibition assay testing MTIC compared with the prodrug YU252215. Whereas MTIC remains inactive in cultured cancer cells and is known to require hydroxylation in the liver to mature to its active form, the prodrug of this invention is activated within the cancer cells and so shows cell growth inhibitory activity.

FIG. 3 is a graph depicting an MDA-MB-231 cell growth inhibition assay testing Cyclophosphamide compared with the prodrug YU252213. Whereas Cyclophosphamide remains inactive in cultured cancer cells and is known to require hydroxylation in the liver to mature to its active form, the prodrug of this invention is activated within the cancer cells and so shows cell growth inhibitory activity.

FIG. 4 is a graph depicting an MDA-MB-231 cell growth inhibition assay testing YU252213 at two different pH levels. Whereas Cyclophosphamide must be activated in the liver prior to exerting its antitumor effect in cancer cells and therefore cannot be coupled with tumor-targeted delivery technologies, the prodrug of this invention is activated within cancer cells and so shows its capability as a warhead for tumor-targeted drug delivery.

FIGS. 5A-5C are graphs depicting PEO1 cell growth inhibition assay testing at two different pH levels, wherein multiple prodrugs of the invention show their capability as warheads for tumor-targeted drug delivery.

### DETAILED DESCRIPTION OF THE INVENTION

In various aspects and embodiments, the invention provides prodrugs that can be reductively cleaved in the cytosol of a tumor cell in a patient, thus originating an active chemotherapy drug.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

The term “abnormal”, when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “biomarker targeting moiety” means a chemical group, structure or biomolecule attached to a therapeutic agent that, when administered to a patient, localizes the therapeutic agent to the tissue presenting the biomarker. By way of non-limiting example, this may be an antibody, a metabolite, or a tumor targeting moiety.

The term “cancer” refers to the physiological condition in a subject typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, “core acid” refers to an acidic group with pKa ranging from about 4.5 to about 7.5.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “DNA targeting moiety” refers to a group that can be conjugated to a chemotherapeutic agent and that, when the conjugate is administered to a patient, help localize the chemotherapeutic agent to or near DNA within a cell. By way of non-limiting example, the DNA targeting moiety is a DNA intercalating agent, a minor-groove binding moiety, a major-groove binding moiety, a phosphate backbone binding moiety, or any combination of these groups.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

An “electron donating group” as used herein refers to an atom or group that adds electron density to neighboring atoms from itself. In certain embodiments, electron donating groups include, but are not limited to, H, alkyl, cycloalkyl, amino, N-alkyl, N-aryl, O-alkyl, and/or O-aryl.

An “electron withdrawing group” as used herein refers to an atom or group of covalently bonded atoms that draws electron density from neighboring atoms towards itself. In certain embodiments, electron withdrawing groups include, but are not limited to, halo, halomethyl, polyhalomethyl, haloalkyl, polyhaloalkyl, phenyl, benzyl, O-phenyl, cyano, carbonyl, carboxyl, ketone, aldehyde, amido, ester, hydroxy, methoxy, ether, alkene, alkyne, thio, thioether, thioester, nitro, nitroso, sulfonamido [—NHS(═O)₂-alkyl, —NHS(═O)₂-aryl, or —S(═O)₂NHR where R can be H, alkyl, or aryl], sulfonate [—OS(═O)₂R′, —S(═O)₂OH, or —S(═O)₂OR′, where R′ can be alkyl or aryl], sulfoxide [—S(═O)R′, where R′ can be alkyl or aryl], and/or sulfone [—S(═O)₂R′, where R′ can be alkyl or aryl].

The terms “MTIC” and “HMMTIC” refer to 3-methyl-(triazen-1-yl)imidazole-4-carboxamide, and 5-(3-hydroxymethyl-3-methyl-1-triazeno)imidazole-4-carboxamide, respectively, or any salts or solvates thereof.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “pH-low insertion peptide” (pHLIP) refers to a water-soluble membrane peptide that interacts weakly with a cell membrane at neutral pH, without insertion into the lipid bilayer; however, at slightly acidic pH (<7.0), pHLIP inserts into the cell membrane and forms a stable transmembrane alpha-helix. pHLIPs are exemplified for example in WO 2012/047354, and U.S. Patent Application Publications No. 20015/0051153 and 2016/0256560, each of which is incorporated by reference herein in its entirety.

As used herein, the term “procure” or “procuring” as relating to a subject in need of being administered a therapeutically active compound refers to the act of synthesizing, packaging, prescribing, purchasing, providing or otherwise acquiring the compound, so that the subject may be administered the compound.

The term “prodrug” refers to a derivatized form of a drug molecule that, while in certain embodiments not pharmacologically active itself, is chemically or enzymatically altered in the body to produce one or more active forms of the drug. A prodrug can in various embodiments be pharmacologically active, but can be chemically or enzymatically altered in the body to produce a more active form or a form with different pharmacological activity.

A “therapeutic” treatment is a treatment administered to a subject who exhibits signs of pathology, for the purpose of diminishing or eliminating those signs.

As used herein, “therapeutic index” refers to the ratio of the toxic dose, or dose of a drug that causes adverse effects incompatible with effective treatment of the disease or condition, to the effective dose, or dose of a drug that leads to the desired therapeutic effect in treatment of the disease or condition.

The phrase “therapeutically effective amount,” as used herein, refers to an amount that is sufficient or effective to prevent or treat (delay or prevent the onset of, prevent the progression of, inhibit, decrease or reverse) a disease or condition associated with cancer, including alleviating symptoms of such diseases.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, the term “tumor targeting moiety” means a group attached to a chemotherapeutic agent that, when administered to a patient, localizes the chemotherapeutic agent to the tumor. By way of non-limiting example, this may be a pHLIP, a cell-penetrating peptide, an antibody, an antibody fragment or antibody mimic, a vitamin or vitamin-mimicking group, a cell-surface receptor-binding small molecule, a weakly-acidic moiety, a polymer subunit, nanoparticle subunit or a group meant to facilitate the incorporation of the compound into a dendrimer, polymer, nanoparticle or liposome, a protein or a protein-binding moiety. In various embodiments the tumor targeting moiety is a weakly acidic group with a pKₐ between about 4.5 and about 7.5. As discussed in PCT/US2018/044164, hereby incorporated by reference, lower pK_{A} values are thought to be more restrictive of drug uptake and to impart more tumor-specific treatment, and higher values are thought to be more permissive of drug uptake and to impart more dose-intensive treatment. In various embodiments, the tumor targeting moiety has a pKₐ outside of the range from 4.5 to 7.5, and/or the acidic/basic character of the tumor targeting moiety is unrelated to its tumor targeting activity.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In one aspect, the invention provides a compound of formula (1):

R-Linker-Drug (1),
which is selected from the group consisting of:

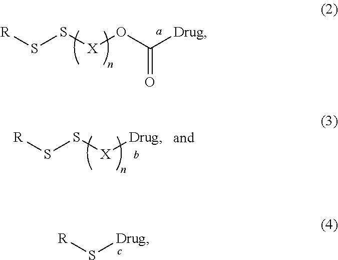

wherein:
Drug is a chemotherapy drug;
each instance of R is independently selected from the group consisting of a biomarker targeting moiety, a tumor targeting moiety, a DNA targeting moiety, C₁-C₆ alkyl, and aryl; each occurrence of n is independently an integer ranging from 1 to 4;
each occurrence of X is independently selected from the group consisting of CH₂, CH(alkyl), and C(alkyl)₂;
bond a is formed between the carbon and a substituent on Drug, wherein the substituent is selected from the group consisting of primary amine, secondary amine, and hydroxyl;
bond b is formed between the carbon and a substituent on Drug, wherein the substituent is selected from the group consisting of hydroxyl, carboxyl, amide, and phosphoramide; and
bond c is formed between the carbon and a substituent on Drug, wherein the substituent is a sulfur atom;
or a salt, solvate, enantiomer, diastereomer, geometric isomer, or tautomer thereof.

In certain embodiments, the compound releases a single active chemotherapeutic agent into the cytosol upon undergoing reductive cleavage therein. In various embodiments, multiple chemotherapeutic agents are released from a single prodrug molecule. Mechanisms by which various embodiments of the herein disclosed compounds can release active chemotherapeutic agents in the cytosol are shown in a non-limiting manner in Examples 1-8.

In certain embodiments, the Drug is 3-methyl-(triazen-1-yl)imidazole-4-carboxamide (MTIC). In various embodiments, the compound of formula (1) is:

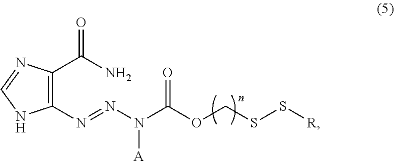

wherein R is defined as above, or a salt, solvate, enantiomer, diastereomer, geometric isomer, or tautomer thereof. In certain embodiments, A is selected from the group consisting of alkyl, haloalkyl, benzyl, halobenzyl, methyl, 2-chloroethyl, and ethyl methanesulfonate. In various embodiments, A is selected from the group consisting of alkyl, haloalkyl, benzyl, and halobenzyl. In yet various embodiments, A is selected from the group consisting of methyl and 2-chloroethyl.

In certain embodiments, the Drug is a pyrrolobenzodiazepine. In various embodiments, the compound of formula (1) is:

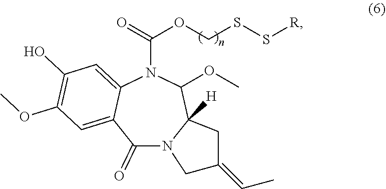

wherein R is defined as above, or a salt, solvate, enantiomer, diastereomer, geometric isomer, or tautomer thereof.

In certain embodiments, the Drug is a nitrogen mustard. In various embodiments, the compound of formula (1) is:

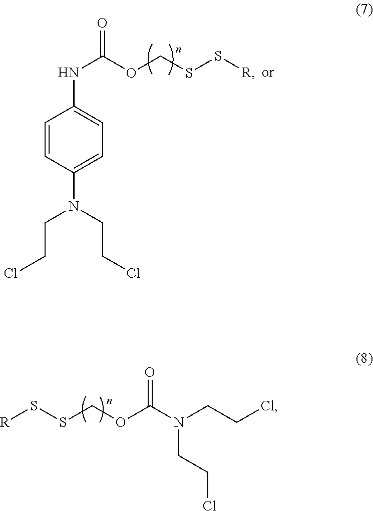

wherein R is defined as above, or a salt, solvate, enantiomer, diastereomer, geometric isomer, or tautomer thereof.

In certain embodiments, the Drug is 12-hydroxyellipticine or 13-hydroxyellipticine.

In various embodiments, the compound of formula (1) is:

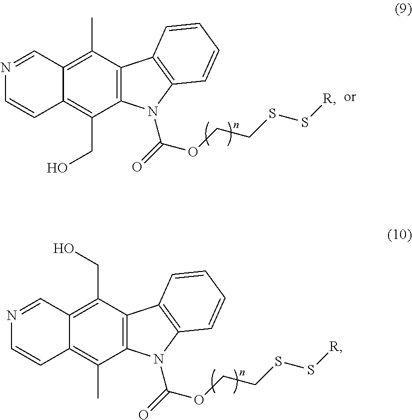

wherein R is defined as above, or a salt, solvate, enantiomer, diastereomer, geometric isomer, or tautomer thereof.

In certain embodiments, the Drug is a an N-nitrosocarbamate. In various embodiments, the compound of formula (1) is:

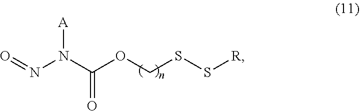

wherein R is defined as above, or a salt, solvate, enantiomer, diastereomer, geometric isomer or tautomer thereof. In certain embodiments A is selected from the group consisting of methyl, ethyl, 2-chloroethyl, and 2-bromoethyl. In various embodiments, A is selected from the group consisting of alkyl, haloalkyl, benzyl, and halobenzyl. In yet various embodiments, A is selected from the group consisting of methyl and 2-chloroethyl.

In various embodiments, the compound of formula (1) is:

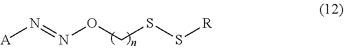

wherein A is selected from the group consisting of alkyl, haloalkyl, benzyl, halobenzyl, methyl, 2-chloroethyl, and ethyl methanesulfonate.

In certain embodiments, the Drug is 5-(3-hydroxymethyl-3-methyl-1-triazeno)imidazole-4-carboxamide (HMMTIC). In various embodiments, the compound of formula (1) is:

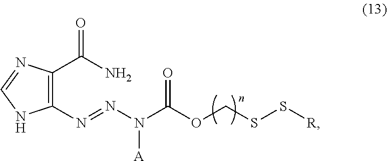

wherein R is defined as above, or a salt, solvate, enantiomer, diastereomer, geometric isomer or tautomer thereof. In certain embodiments A is selected from the group consisting of alkyl, haloalkyl, benzyl, and halobenzyl, methyl, 2-chloroethyl, and ethyl methanesulfonate. In various embodiments, A is selected from the group consisting of alkyl, haloalkyl, benzyl, and halobenzyl. In various embodiments, A is selected from the group consisting of methyl and 2-chloroethyl.

In certain embodiments, the Drug is a phosphoramide mustard. In various embodiments, the compound of formula (1) is:

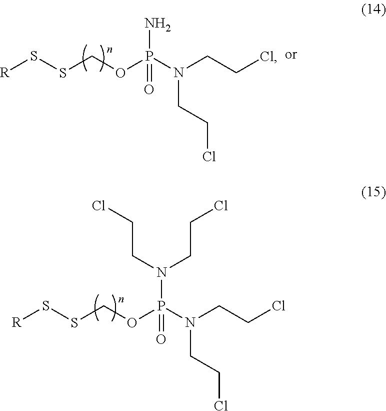

wherein R is defined as above, or a salt, solvate, enantiomer, diastereomer, geometric isomer or tautomer thereof.

In certain embodiments, the Drug is o-thioquinone methide. In various embodiments, the compound of formula (1) is:

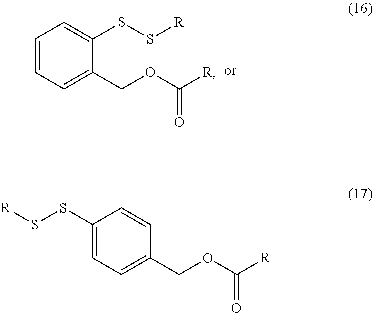

wherein each independent instance of R is defined as above, or a salt, solvate, enantiomer, diastereoisomer, geometric isomer or tautomer thereof.

In another aspect, the invention provides a compound of formula (18):

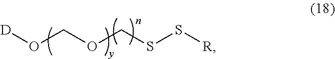

wherein:
D is a DNA binding or nuclear localizing moiety,
R is selected from the group consisting of a biomarker targeting moiety, a tumor targeting moiety, a DNA targeting moiety, C₁-C₆ alkyl, and aryl,
each occurrence of n is independently an integer ranging from 1 to 4,
y is 0-20;
or a salt, solvate, enantiomer, diastereoisomer, geometric isomer or tautomer thereof. In various embodiments, y is 2.

In various embodiments, any of the above described compounds containing substituent R wherein R is a weakly acidic group having a pKₐ between about 4.5 and about 7.5.

In various embodiments, R is selected from the group consisting of:

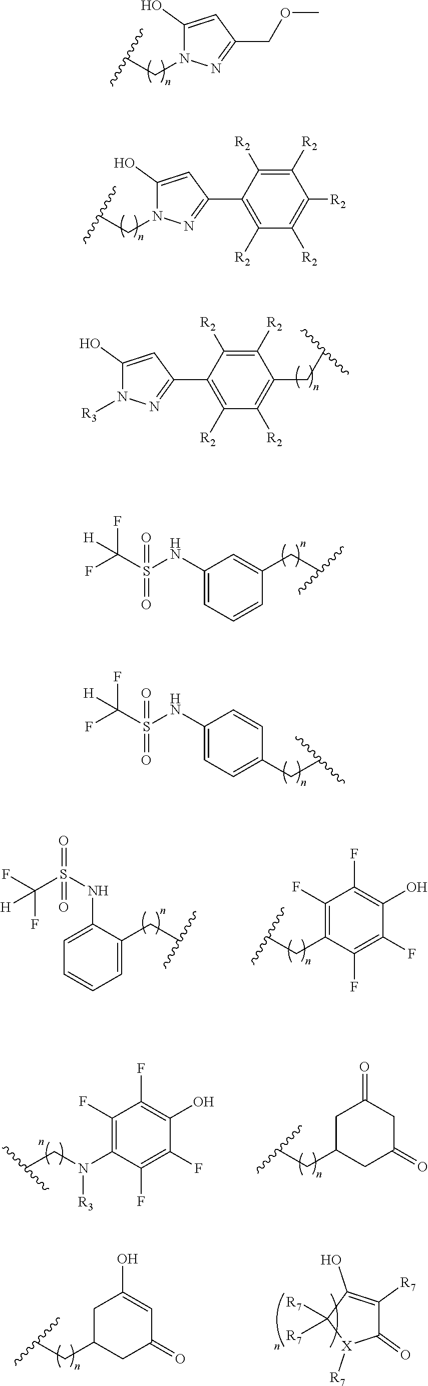

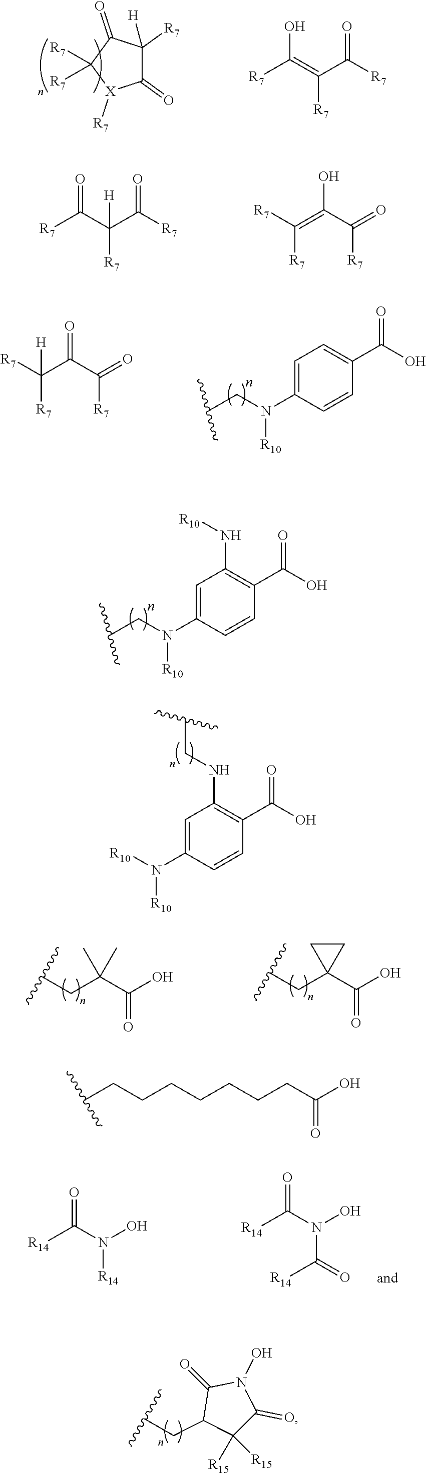

wherein:
each instance of R₂ is independently selected from the group consisting of H, F, Cl, hydroxy, methoxy, —NH₂, —NH-alkyl, —N(alkyl)₂, and alkyl;
R₃ is selected from the group consisting of H, methyl, ethyl, alkyl, phenyl, benzyl, haloaryl, —CH₂—O—CH₃, and —CH₂—CH₂—OH;
each instance of R₇ is independently selected from the group consisting of H, alkyl, phenyl, benzyl, an electron donating group, or a covalent bond to linker or drug;
X is CH, C-alkyl, or N;
at least one R₇ group comprises a covalent bond to Linker or Drug either directly or by displacing a hydrogen on alkyl, phenyl, benzyl or an electron donating group;
each instance of R₁₀ is independently selected from the group consisting of H, alkyl, or an electron donating group;
each instance of R₁₄ is independently an electron withdrawing group, an electron donating group or a covalent bond to Linker or Drug;
at least one R₁₄ group comprises a covalent bond to Linker or Drug either directly or by displacing a hydrogen on an electron withdrawing or electron donating group;
each instance of R₁₅ is independently selected from the group consisting of: H, an electron withdrawing group or an electron donating group; and
n is an integer ranging from 0 to 4.

In various embodiments, the compound is selected from the group consisting of:

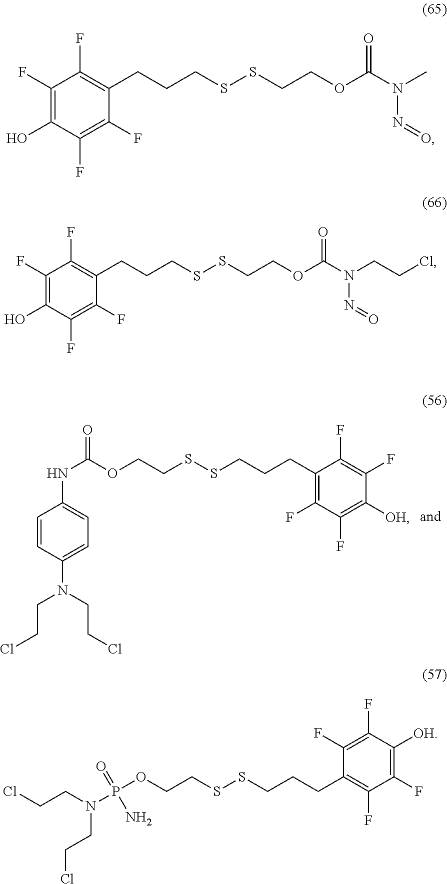 [SMILES: CN(N=O)C(=O)OCCSSCCCc1c(F)c(F)c(O)c(F)c1F ; O=NN(CCCl)C(=O)OCCSSCCCc1c(F)c(F)c(O)c(F)c1F ; O=C(Nc1ccc(N(CCCl)CCCl)cc1)OCCSSCCCc1c(F)c(F)c(O)c(F)c1F ; NP(=O)(OCCSSCCCc1c(F)c(F)c(O)c(F)c1F)N(CCCl)CCCl]

The compounds of the invention may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In various embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol, or buffered solutions thereof. In various embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into an active therapeutic compound in vivo. In certain embodiments, upon in vivo administration, a prodrug is chemically converted to the biologically, pharmaceutically or therapeutically active form of the compound. In various embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In various embodiments, substitution with heavier isotopes such as deuterium affords greater metabolic stability (for example, increased in vivo half-life or reduced dosage requirements). In various embodiments, substitution with positron emitting isotopes, such as ¹¹C, ¹⁸F, ¹⁵O and ¹³N, is useful in Positron Emission Topography (PET) studies for examining biodistribution or substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

### Drugs

In certain embodiments, the drug is a chemotherapeutic drug, which has cytotoxic and/or anticancer activity. In various embodiments, the drug comprises, or can be derivatized to comprise, a primary amine, secondary amine, a hydroxyl, or a thiol. A person of skill in the art will recognize that the disclosure may be applied to chemotherapeutic drugs of known efficacy, as well as compounds which efficacy has not previously been appreciated.

The drug may, but is not limited to, exert its primary antitumor activity through: alkylating activity, by way of non-limiting example, a nitrogen mustard; or other mechanisms known in the art to achieve antitumor activity in vivo.

In certain embodiments, the linker acts as a sensor for cell insertion, responding to the reductive environment of the cytosolic compartment inside a cell by allowing for traceless release of the drug. In various embodiments, the covalent linker is more stable in blood than in the cytosol of a tumor cell, and/or undergoes cleavage and/or spontaneous rearrangement in the cytosolic compartment of cells so as to release the active drug. In various embodiments, the covalent linker is relatively stable outside of cells.

A person of ordinary skill in the art will recognize, in particular from the examples herein, that the drug may be an agent that, absent the modifications described herein, would be too toxic to be administered to patients or that would be limited to low doses by the toxicity of the compound. Without wishing to be limited by theory, these compounds are rendered non-toxic or their toxicity is reduced in the blood or other extracellular space where the linker remains attached Once within the tumor, the linker is removed releasing the active cytotoxic agent. Accordingly, in some embodiments, this “active form” of the compound is only released upon reduction of the linker in the cytosol.

### Pharmaceutical Compositions

In certain embodiments, the compounds herein disclosed herein can be formulated as a pharmaceutical composition comprising at least one pharmaceutically acceptable excipient.

In various embodiments, the composition further comprises at least one additional chemotherapeutic drug.

The pharmaceutical composition can be formulated for administration by any route deemed appropriate by a person of skill in the art based on the properties of the drug and the needs of the patient. In certain embodiments, the pharmaceutical composition is formulated for nasal, inhalational, topical, oral, buccal, rectal, pleural, peritoneal, vaginal, intramuscular, subcutaneous, transdermal, epidural, intratracheal, otic, intraocular, intrathecal or intravenous administration.

Further details regarding formulation, administration and dose are discussed elsewhere herein.

### Methods of Treatment

The present invention includes methods for treating and/or preventing of cancer. In another aspect, the invention provides a method for treating a cancer in a subject in need thereof, the method comprising administering to the subject a therapeutically effective amount of the above described compounds or pharmaceutical compositions.

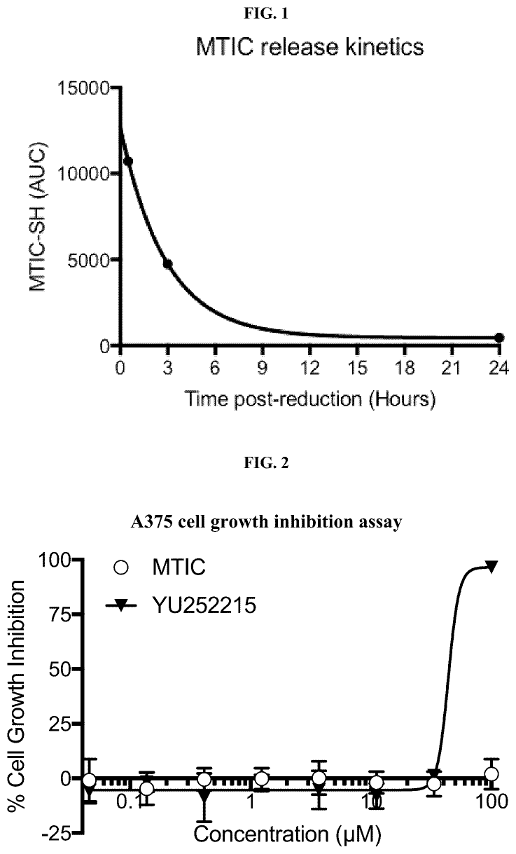

Without wishing to be limited by theory, the prodrug form of the compound circulates in the extracellular space, until entering the cytosol and reductive cleavage of the linker and/or disulfide generates the active form of the prodrug. Examples of the reaction or series of reactions that take place in connection with various embodiments of the invention are illustrated elsewhere herein. The release kinetics of a prodrug of MTIC are exemplified in a non-limiting manner in FIG. 1.

In certain embodiments the cancer is at least one selected from the group consisting of melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-Hodgkins lymphoma, hepatocellular carcinoma, bladder cancer, and lung cancer.

In certain embodiments, the method further comprises procuring the compound or the pharmaceutical composition for the subject. In various embodiments, the method further comprises administering to the subject additional cancer treatment. By way of non-limiting example, the additional cancer treatment may include but is not limited to radiation, surgical excision, immunotherapy, and antiproliferative chemotherapy.

For example, prodrug compounds of the invention can be administered in combination with one or more additional chemotherapeutic agents. As used herein, two agents are said to be administered in combination when the two agents are administered simultaneously, or are administered independently in a fashion such that the agents act at the approximately same time.

The dosage administered will be dependent upon the age, health, and weight of the recipient, kind of concurrent treatment, if any, frequency of treatment, and the nature of the effect desired.

In some embodiments, the above described compounds or pharmaceutical compositions are provided in a prepackaged pharmaceutical composition with at least one instructional material for use thereof, wherein the instructional material comprises instructions for preventing or treating cancer in a subject.

### Combination Therapies

The compounds useful within the methods of the invention may be used in combination with one or more additional therapeutic agents useful for treating a cancer. These additional therapeutic agents may comprise compounds that are commercially available or synthetically accessible to those skilled in the art. These additional therapeutic agents may be known to treat, prevent, or reduce the symptoms, of a cancer.

In certain embodiments, administering the prodrug compound of the invention to the subject allows for administering a lower dose of the additional therapeutic agent as compared to the dose of the additional therapeutic agent alone that is required to achieve similar results in treating or preventing a disease or disorder in the subject. For example, in certain embodiments, the prodrug compound of the invention enhances the anticancer activity of the additional therapeutic compound, thereby allowing for a lower dose of the additional therapeutic compound to provide the same effect.

In certain embodiments, the compounds of the present invention are used in combination with radiation therapy. In various embodiments, the combination of administration of the compounds of the present invention and application of radiation therapy is more effective in treating or preventing cancer than application of radiation therapy by itself. In various embodiments, the combination of administration of the compounds of the present invention and application of radiation therapy allows for use of lower amount of radiation therapy in treating the subject.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a cancer in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In various embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other therapeutic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Application Publication Nos. 2003/0147952; 2003/0104062; 2003/0104053; 2003/0044466; 2003/0039688; and 2002/0051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation. In another embodiment, sustained release may be achieved by encapsulation of the compounds within biocompatible microstructures, such as liposomes, micelles or nanoparticles.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a cancer in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between the clinically effective dose and the maximum tolerable dose (MTD) or the side effect inducing dose. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Herein, each instance of R is independently selected from the group consisting of a biomarker targeting moiety, a tumor targeting moiety, a DNA targeting moiety, C₁-C₆ alkyl, and aryl, as defined above.

### Example 1: DNA-Targeted Reductively Activated Paraformaldehyde Chemotherapy

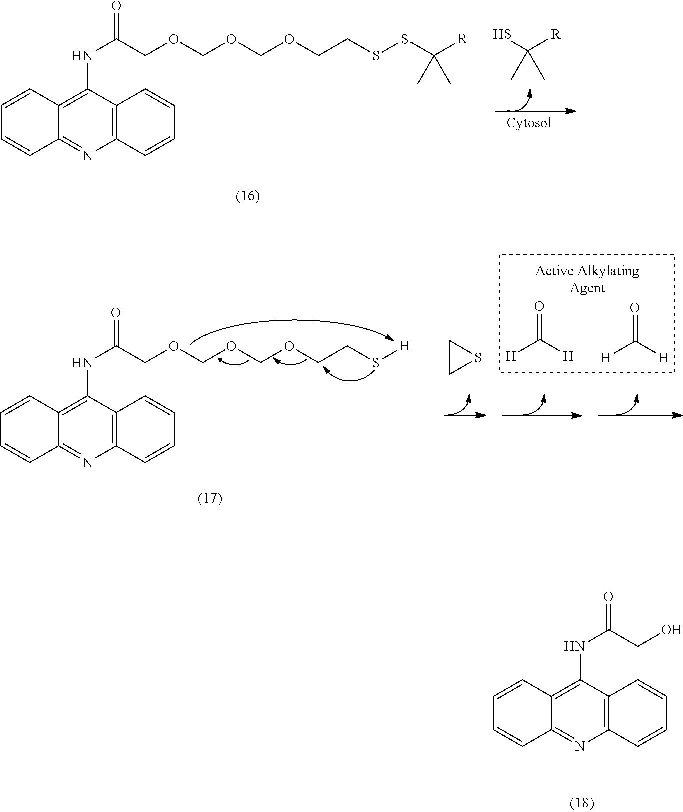

### Example 2: Reductively Activated MTIC or HMMTIC

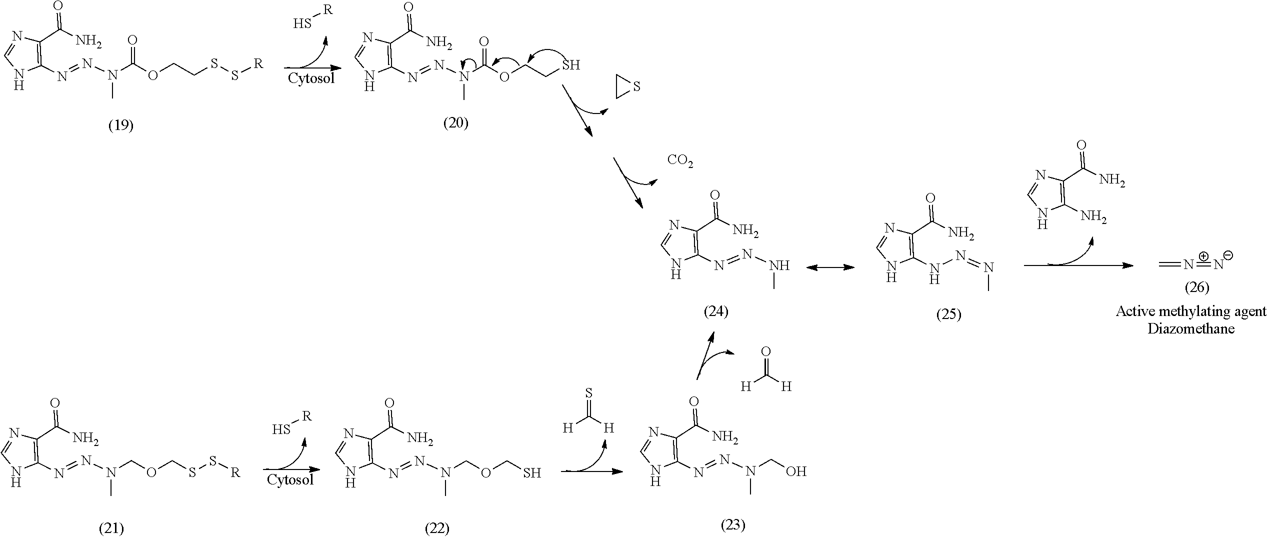

### Example 3: Reductively Activated Pyrrolobenzodiazepines

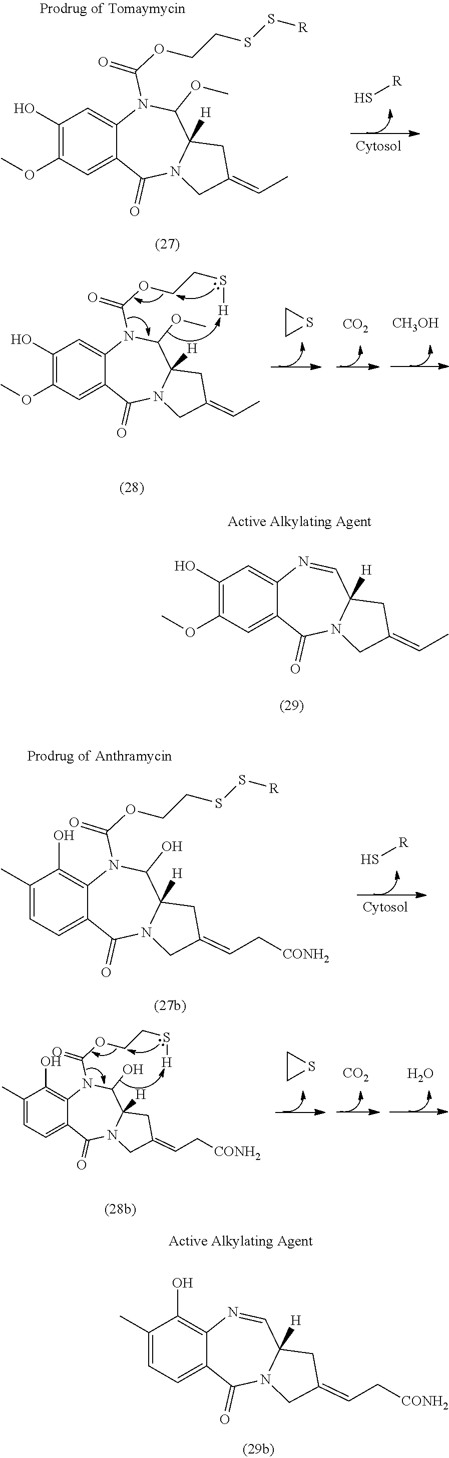

### Example 4: Reductively Activated Nitrogen Mustards

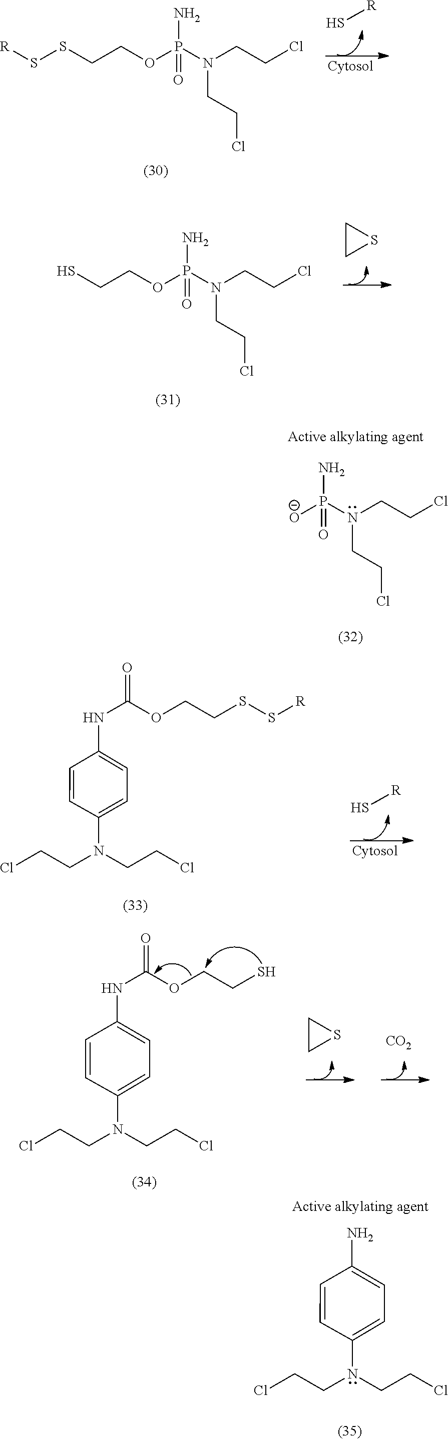

### Example 5: Reductively Activated Ellipticine and its Hydroxylated Intermediates

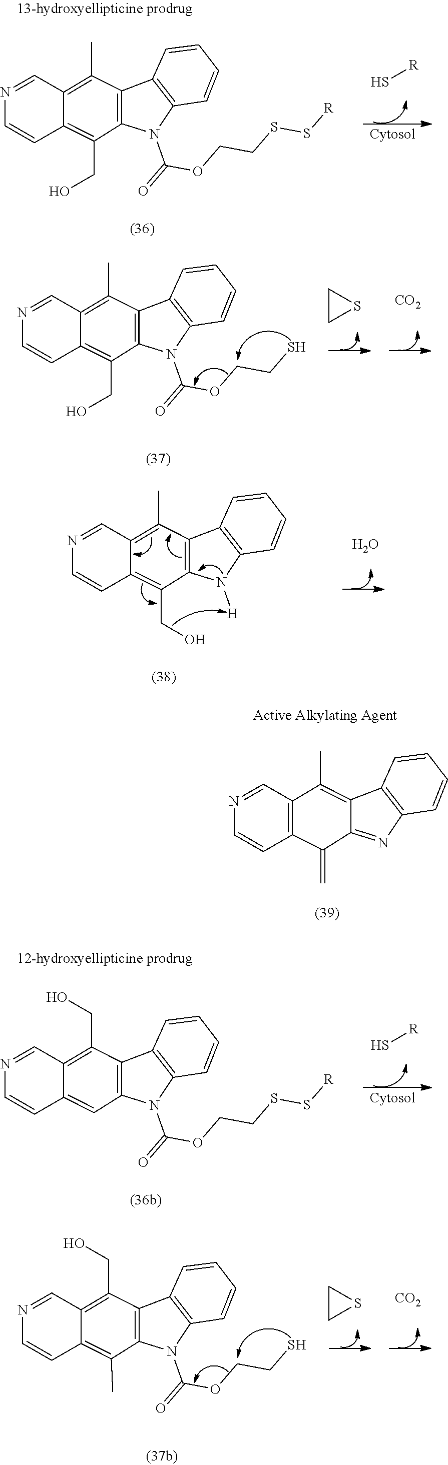

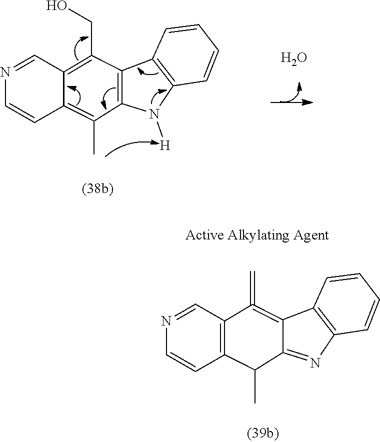

### Example 6: Reductively Activated N-Nitrosocarbamates

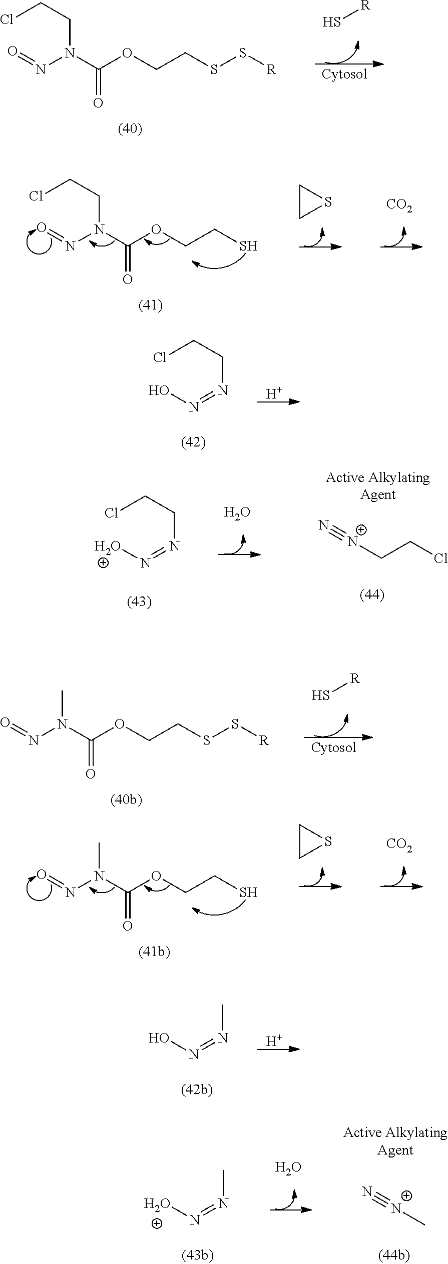

### Example 7: Reductively Activated o-Thioquinone Methide

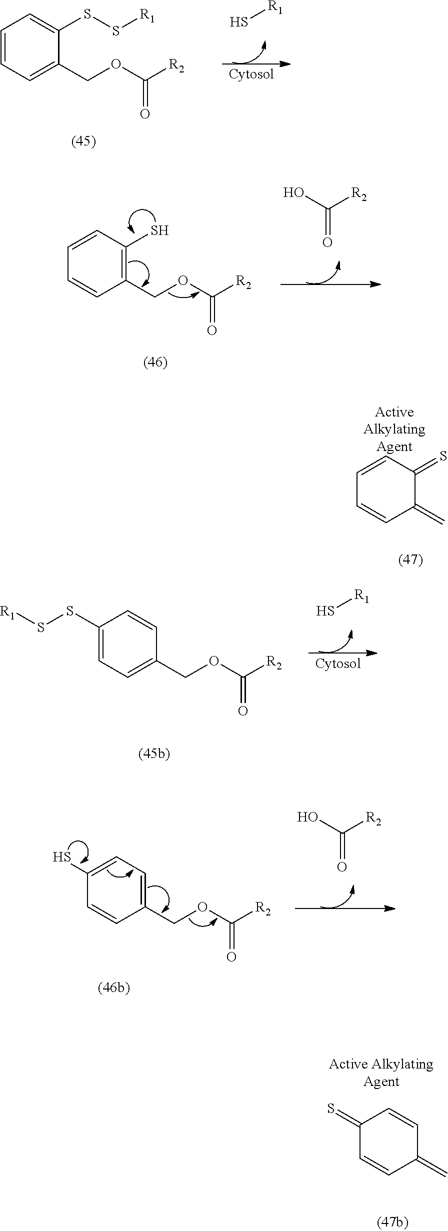

### Example 8: In Vitro Studies

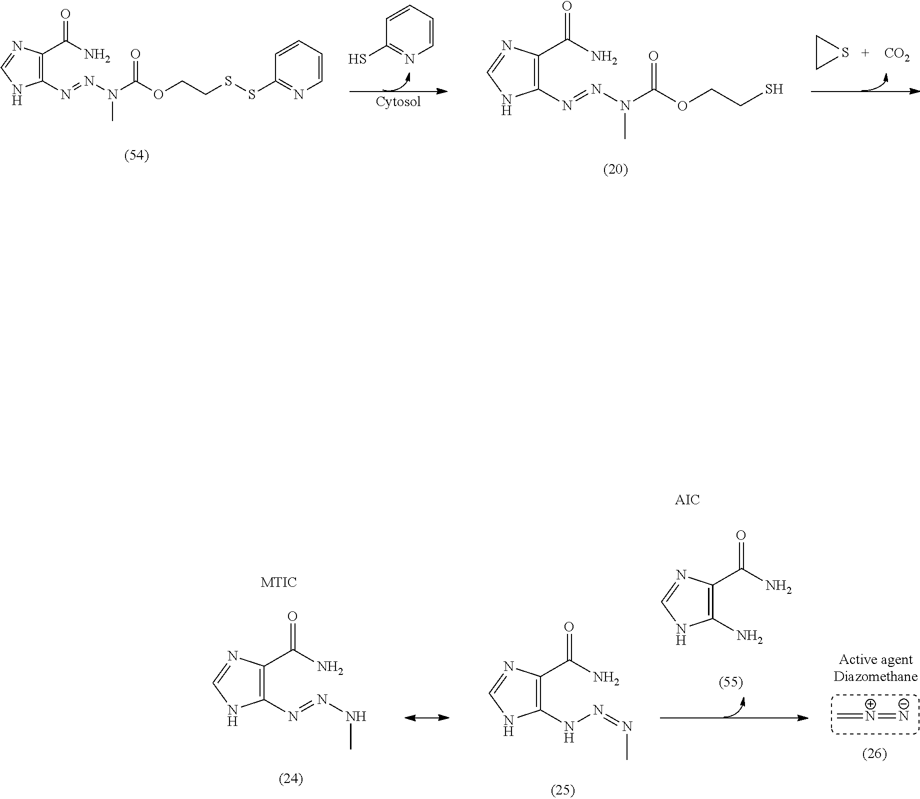

This mechanism has been tested in vitro and the prodrug, YU252215 (54) was found to be highly stable in non-reductive aqueous conditions. Using LCMS, (54) was monitored in the presence of reducing reagents, such as glutathione (GSH) and dithiothreitol (DTT). Within minutes of addition of the reducing agent, (54) was no longer detectable and the free thiol (20) predominated. This led over time to maturation to MTIC (24), which was detected but rapidly progressed to release the active alkylating agent diazomethane (26), which was not directly detected. However, the side product of diazomethane release AIC (55) was detected in concert with the appearance and subsequent disappearance of MTIC (24). The kinetics of MTIC production were roughly calculated from the decrease in area under the curve of the thiol (20) as a function of time, giving t_{1/2}=1.99 hours (FIG. 1), which agrees closely with the half-life measured for doxorubicin release from this same linker structure.

MTIC (24) is highly reactive in aqueous environments. In certain non-limiting embodiments, it is not therapeutically useful itself, as it rapidly and spontaneously progresses to release its potentially therapeutic DNA-alkylating agent (26), which reacts immediately with water or physiological solutes in the blood prior to reaching the cells of the tumor that are its intended therapeutic target. MTIC (24) is produced as the activated intermediate of two approved chemotherapeutic prodrugs, temozolomide and dacarbazine. Temozolomide progresses to (24) spontaneously in physiological conditions, such as the blood, while dacarbazine is stable until acted upon by cytochrome P450 enzymes, predominantly in the liver, to produce HMMTIC (23), which spontaneously progresses to (24) in physiological conditions, such as the blood. Both existing approved prodrugs of MTIC (24) are predominantly activated outside of the tumor tissue that is their therapeutic target. The mechanism by which YU252215 (54) and similar prodrugs utilizing this approach, (19) and (21), are activated to release (24) is dependent upon cytosolic reducing agents. In a cell growth inhibition assay, in vitro, wherein treatments were administered transiently for 6 hours, then replaced with growth medium for the growth phase of the experiment, (54) demonstrates the increased therapeutic potential of this prodrug mechanism over MTIC (24) alone. Cytotoxicity of (54) against EMT-6 mammary cancer cells was detected at an IC₅₀ value of around 50 μM (FIG. 2), whereas MTIC (24) showed no toxicity in this assay. This mechanism therefore retains a greater proportion of the therapeutic potential of (24) on its way inside of targeted cells, and can further be coupled with targeted delivery strategies.

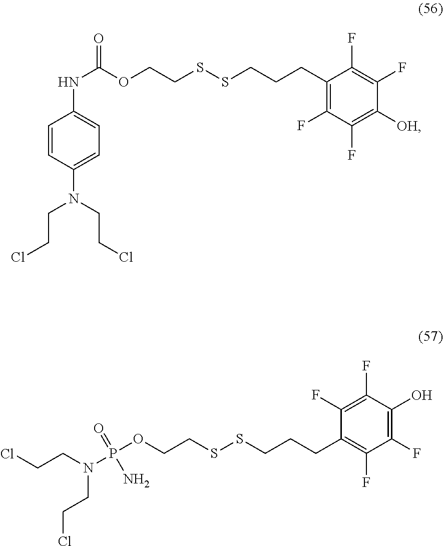 [SMILES: O=C(Nc1ccc(N(CCCl)CCCl)cc1)OCCSSCCCc1c(F)c(F)c(O)c(F)c1F ; NP(=O)(OCCSSCCCc1c(F)c(F)c(O)c(F)c1F)N(CCCl)CCCl]

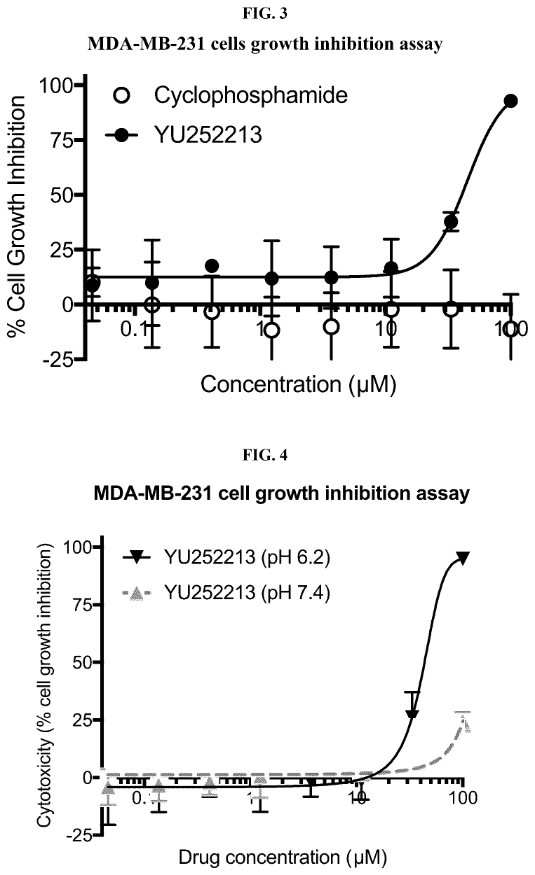

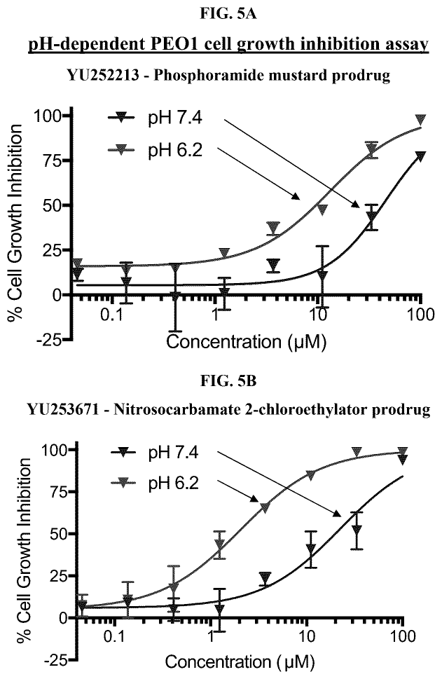

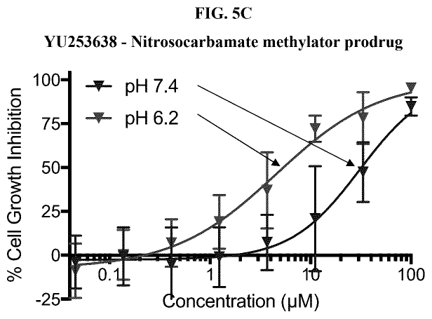

Examples of nitrogen mustards (30) and (33) were also shown in vitro to achieve cytosolically activated cytotoxic activity, coupled with an R-group designed to achieve targeted delivery. The cytotoxicity of two pH-dependent nitrogen mustard prodrugs, YU252353 (56) and YU252213 (57), designed to selectively permeate and therefore selectively treat cells in acidic extracellular tumor environments, was evaluated in MDA-MB-231 breast cancer cells and showed preferential cytotoxicity in acidic conditions mimicking the tumor microenvironment (pH 6.2) over that in normal physiological pH 7.4. In MDA-MB-231 cells, YU252213 (57) was measured to have IC₅₀ values of 41.4 μM at tumor pH 6.2 and >100 μM at healthy tissue pH 7.4 (FIG. 3). In EMT-6 cells, YU252353 (56) was measured to have an IC₅₀ value of 28.7 μM at tumor pH 6.2, and had no detectable toxicity in this assay at normal physiological pH 7.4 (FIG. 4).

### Example 9: Synthetic Examples

Compounds (58), (59), and (60) were synthesized according to the reported procedure or using methods known in the art: (58) (Chem. Eur. J. 2006, 12, 3655-3671), (59) (Bioorg. Med. Chem. 2004, 12, 771-777), (60) (Chem. Eur. J. 2006, 12, 3655-3671).

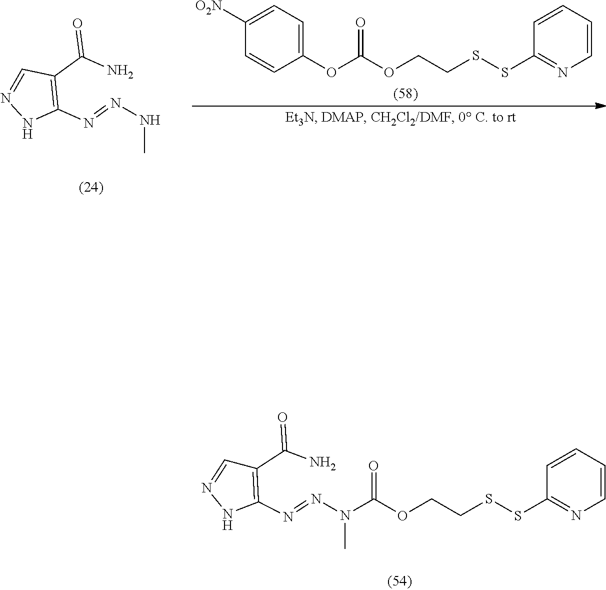 [SMILES: CN/N=N/c1[nH]ncc1C(N)=O ; O=C(OCCSSc1ccccn1)Oc1ccc([N+](=O)[O-])cc1 ; CN(/N=N/c1[nH]ncc1C(N)=O)C(=O)OCCSSc1ccccn1]

MTIC-SS-Py (54): To 3-Methyl-(triazen-1-yl)imidazole-4-carboxamide (24) (40 mg, 0.24 mml) in CH₂Cl₂/DMF (2:1) 1.5 mL were added (58) (92 mg, 0.26 mmol), triethyl amine (52 μL, 0.36 mmol) and DMAP (0.05 mmol, 6 mg) at 0° C. The reaction mixture was allowed warm up to room temperature, and stirring was continued for overnight. Solvents were removed by rotary evaporator and purified by silica gel column chromatography (CH₂Cl₂:MeOH) to provide MTIC-SS-Py (54) as a light gray solid (9 mg, 10% yield). ¹H NMR (500 MHz, CDCl₃) δ 8.48 (d, J=4.7 Hz, 1H), 8.20 (s, 2H), 7.93 (s, 1H), 7.64 (d, J=3.8 Hz, 2H), 7.09 (q, J=4.7 Hz, 1H), 4.57 (m, 2H), 3.33 (s, 3H), 3.16 (t, J=6.5 Hz, 2H); LC-MS: (M+H)⁺=382.30 (experimental), 382.07 (calculated).

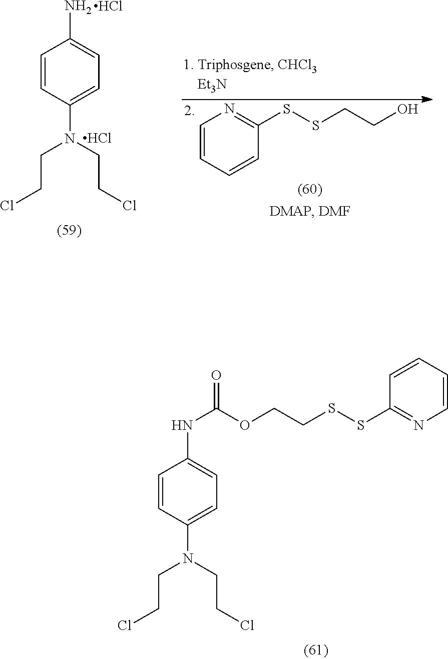 [SMILES: Nc1ccc(N(CCCl)CCCl)cc1 ; Cl ; Cl ; OCCSSc1ccccn1 ; O=C(Nc1ccc(N(CCCl)CCCl)cc1)OCCSSc1ccccn1]

(61): To mustard dihydrochloride (59) (500 mg, 1.63 mmol) in CHCl₃ (5 mL) at 0° C. was added triethylamine (0.7 mL, 4.9 mmol). The solution was cooled to −20° C. and triphosgene (384 mg, 1.3 mmol) in chloroform (2 mL) was added. The solution was removed from cooling and maintained at ambient temperature for 2 h. The solvent was evaporated, and the residue was redissolved in THF (10 mL). To the suspension (60) (322 mg, 1.79 mmol), DMAP (400 mg, 3.26 mmol) was added at the room temperature. The reaction mixture was stirred at room temperature for overnight and added water. The aqueous layer was separated and extracted with EtOAc (3×). The combined organic layer was washed with brine and dried over anhydrous Na₂SO₄. The filtrate was concentrated to dryness and purified by flash column chromatography (SiO₂, Hexane/EtOAc=1:1) to afford compound (61) as brownish oil (365 mg, 50% yield). ¹H NMR (400 MHz, CDCl₃) δ 8.43 (ddd, J=4.9, 1.9, 0.9 Hz, 1H), 7.66 (dt, J=8.1, 1.1 Hz, 1H), 7.59 (td, J=7.7, 1.8 Hz, 1H), 7.22 (d, J=8.4 Hz, 2H), 7.05 (ddd, J=7.3, 4.8, 1.1 Hz, 1H), 6.91 (s, 1H), 6.63-6.55 (m, 2H), 4.35 (t, J=6.3 Hz, 2H), 3.73-3.61 (m, 4H), 3.60-3.50 (m, 4H), 3.04 (t, J=6.3 Hz, 2H); ¹³C NMR (101 MHz, CDCl₃) δ 159.72, 153.58, 149.64, 142.75, 137.11, 128.46, 121.38, 120.88, 119.84, 112.73, 62.72, 53.65, 40.60, 37.84; LC-MS: (M+H)⁺=446.28 (experimental), 446.05 (calculated).

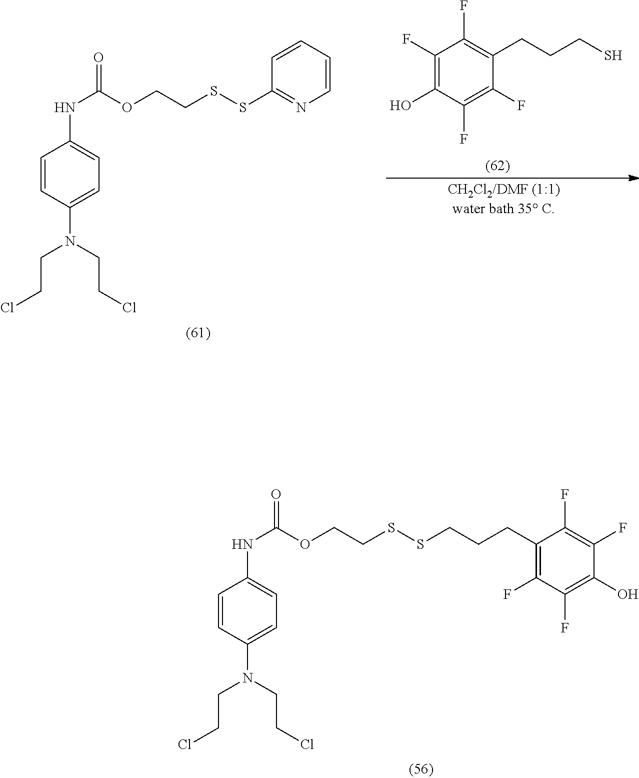 [SMILES: O=C(Nc1ccc(N(CCCl)CCCl)cc1)OCCSSc1ccccn1 ; Oc1c(F)c(F)c(CCCS)c(F)c1F ; O=C(Nc1ccc(N(CCCl)CCCl)cc1)OCCSSCCCc1c(F)c(F)c(O)c(F)c1F]

(56): Aniline mustard-SS-Py (61) (20 mg, 0.04 mmol) and compound (62) (19 mg, 0.08 mmol) in CH₂Cl₂/DMF (1:1, 1 mL) were stirred at 35° C. water bath for 24 hours. The solvent was removed under vacuum and the residue was purified by silica gel column chromatography (Hexanes:EtOAC=1:1) to provide compound (56) (12 mg, 52% yield). ¹H NMR (400 MHz, CDCl₃) δ 7.99 (s, 2H), 6.73-6.56 (m, 2H), 4.36 (t, J=6.6 Hz, 2H), 3.67 (t, J=6.5 Hz, 4H), 3.58 (t, J=7.2 Hz, 4H), 2.94-2.88 (m, 2H), 2.74-2.68 (m, 2H), 1.95 (p, J=7.5 Hz, 3H); ¹³C NMR (101 MHz, CDCl₃) δ 162.77, 149.79, 137.85, 129.98, 123.88, 121.46, 112.77, 65.74, 53.70, 40.48, 38.10, 36.61, 31.54, 28.73, 20.93; ¹⁹F NMR (376 MHz, CDCl₃) δ −146.30 to −146.42 (m, 2F), −163.50 to −163.60 (m, 2F); LC-MS: (M+H)⁺=575.22 (experimental), 577.17 (M+3), 575.06 (calculated).

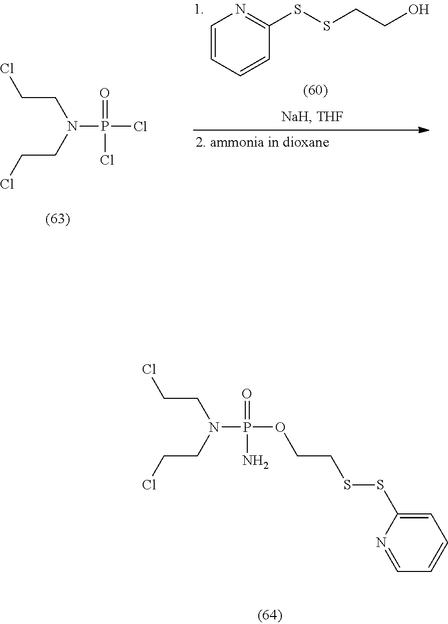 [SMILES: O=P(Cl)(Cl)N(CCCl)CCCl ; OCCSSc1ccccn1 ; NP(=O)(OCCSSc1ccccn1)N(CCCl)CCCl]

(64): To compound (60) (200 mg, 1.07 mmol) in THF (3 mL) was added NaH (60% in mineral oil, 51 mg, 1.30 mmol) at 0° C. The suspension was stirred for 30 min at 0° C. and (63) (276 mg, 1.06 mmol) in 2 mL THF was added. The reaction mixture was warmed to room temperature, and stirring was continued for 4 h at room temperature. The solvent was removed by rotary evaporator, the crude oil was dissolved in dioxane (2 mL) and excess of ammonia in dioxane (0.5 M) was added at room temperature. The stirring was continued for overnight, the dioxane was evaporated and the residue was purified by silica gel column chromatography (CH₂Cl₂:MeOH=9:1) to provide compound (64) (29 mg, 7% yield). ¹H NMR (500 MHz, CDCl₃) δ 8.46-8.40 (m, 1H), 7.69-7.58 (m, 2H), 7.07 (ddd, J=6.7, 4.8, 2.0 Hz, 1H), 4.30-4.07 (m, 2H), 3.58 (t, J=7.1 Hz, 4H), 3.46-3.35 (m, 4H), 3.03 (t, J=6.3 Hz, 2H); ¹³C NMR (126 MHz, CDCl₃) δ 159.34, 149.69, 137.17, 121.02, 120.08, 63.21, 63.17, 49.22, 49.18, 42.51, 39.06, 39.01; ³¹P NMR (202 MHz, CDCl₃) δ 16.16; LC-MS: (M+H)⁺=390.31 (experimental), 390.00 (calculated).

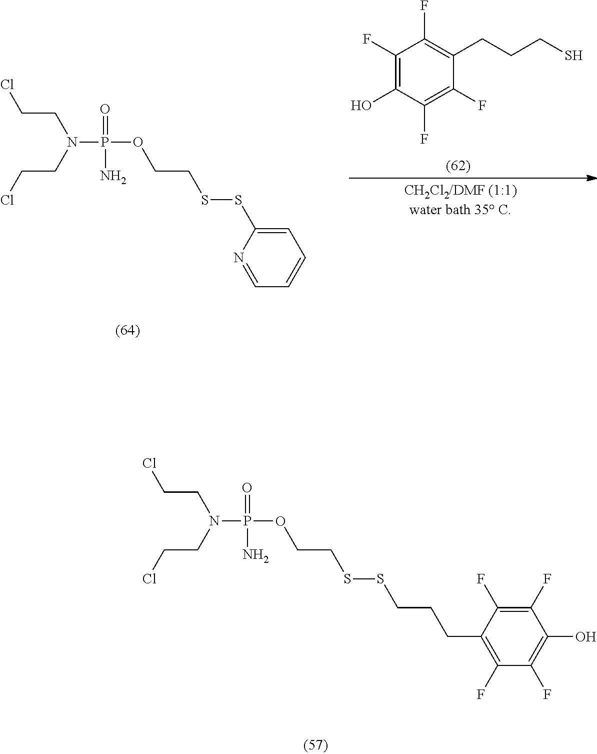 [SMILES: NP(=O)(OCCSSc1ccccn1)N(CCCl)CCCl ; Oc1c(F)c(F)c(CCCS)c(F)c1F ; NP(=O)(OCCSSCCCc1c(F)c(F)c(O)c(F)c1F)N(CCCl)CCCl]

(57): Compound (64) (25 mg, 0.06 mmol) and compound (62) (31 mg, 0.13 mmol) were dissolved in CH₂Cl₂/DMF (1:1) 1 mL. The reaction was stirred at 35° C. water bath for 24 hours. The solvent was removed under vacuum and purified by silica gel column chromatography (Hexanes:EtOAC=2:3) to provide compound (57) (13 mg, 42% yield). ¹H NMR (500 MHz, CDCl₃) δ 4.29-4.07 (m, 2H), 3.72-3.57 (m, 4H), 3.54-3.38 (m, 4H), 2.90 (td, J=6.6, 2.2 Hz, 2H), 2.78-2.63 (m, 4H), 1.95 (t, J=7.4 Hz, 2H); ³¹P NMR (202 MHz, CDCl₃) δ 15.77; ¹⁹F NMR (471 MHz, CDCl₃) δ −146.84 (dd, J=21.6, 8.2 Hz, 2F), −161.33- to −164.94 (m, 2F); HRMS: (M+H)⁺=519.0028 (experimental), 519.0117 (calculated).

The disclosures of each and every patent, patent application, and publication cited herein are hereby incorporated herein by reference in their entirety.

While this invention has been disclosed with reference to specific embodiments, it is apparent that various embodiments and variations of this invention may be devised by others skilled in the art without departing from the true spirit and scope of the invention. The appended claims are intended to be construed to include all such embodiments and equivalent variations.

1. A compound of formula (1):
R-Linker-Drug (1),
which is selected from the group consisting of: 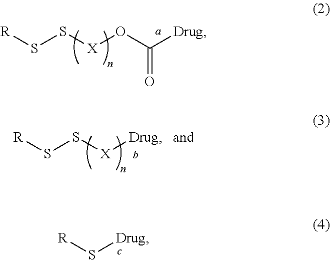
wherein:
Drug is a chemotherapy drug;
each instance of R is independently selected from the group consisting of a biomarker targeting moiety, a tumor targeting moiety, a DNA targeting moiety, C₁-C₆ alkyl, and aryl;
each occurrence of n is independently an integer ranging from 1 to 4;
each occurrence of X is independently selected from the group consisting of CH₂, CH(alkyl), and C(alkyl)₂;
bond a is formed between the carbon and a substituent on Drug, wherein the substituent is selected from the group consisting of primary amine, secondary amine, and hydroxyl;
bond b is formed between the carbon and a substituent on Drug, wherein the substituent is selected from the group consisting of hydroxyl, carboxyl, amide, and phosphoramide; and
bond c is formed between the carbon and a substituent on Drug, wherein the substituent is a sulfur atom;
or a salt, solvate, enantiomer, diastereomer, geometric isomer, or tautomer thereof.

2. The compound of claim 1, wherein the Drug is 3-methyl-(triazen-1-yl)imidazole-4-carboxamide (MTIC), or a salt or solvate thereof.

3. The compound of claim 2, wherein the compound of formula (1) is: 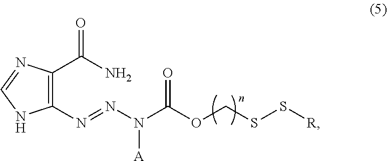
wherein A is selected from the group consisting of alkyl, haloalkyl, benzyl, halobenzyl, methyl, 2-chloroethyl, and ethyl methanesulfonate,
or a salt, solvate, enantiomer, diastereomer, geometric isomer or tautomer thereof.

4. The compound of claim 1, wherein the Drug is a Pyrrolobenzodiazepine.

5. The compound of claim 4, wherein the compound of formula (1) is: 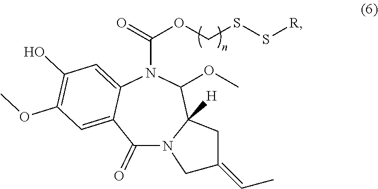
or a salt, solvate, enantiomer, diastereomer, geometric isomer or tautomer thereof.

6. The compound of claim 1, wherein the Drug is a nitrogen mustard.

7. The compound of claim 6, wherein the compound of formula (1) is: 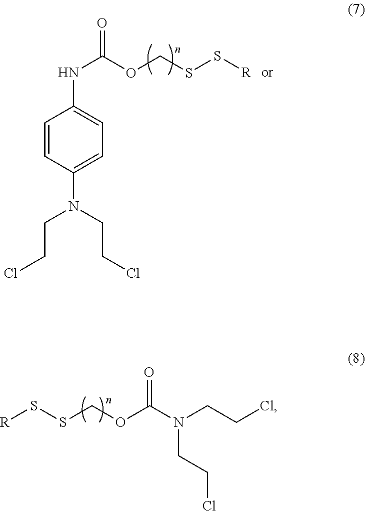
or a salt, solvate, enantiomer, diastereomer, geometric isomer or tautomer thereof.

8. The compound of claim 1, wherein the Drug is 12-hydroxyellipticine or 13-hydroxyellipticine.

9. The compound of claim 8, wherein the compound of formula (1) is: 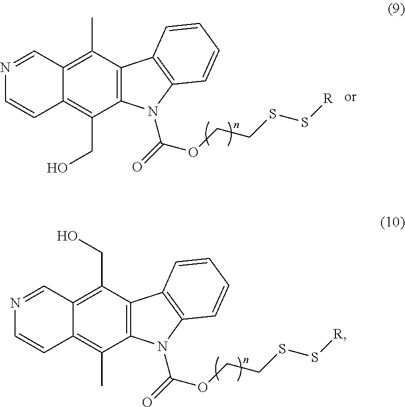
or a salt, solvate, enantiomer, diastereomer, geometric isomer or tautomer thereof.

10. The compound of claim 1, wherein the Drug is a nitrosocarbamate.

11. The compound of claim 10, wherein the compound of formula (1) is: 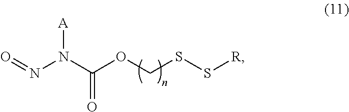
wherein A is selected from the group consisting of alkyl, haloalkyl, benzyl, halobenzyl, methyl, 2-chloroethyl, and ethyl methanesulfonate,
or a salt, solvate, enantiomer, diastereomer, geometric isomer or tautomer thereof.

12. The compound of claim 1, wherein the compound of formula (1) is: 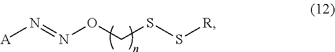
wherein A is selected from the group consisting of alkyl, haloalkyl, benzyl, halobenzyl, methyl, 2-chloroethyl, and ethyl methanesulfonate,

13. The compound of claim 1, wherein the Drug is 5-(3-hydroxymethyl-3-methyl-1-triazeno)imidazole-4-carboxamide (HMMTIC).

14. The compound of claim 13, wherein the compound of formula (1) is: 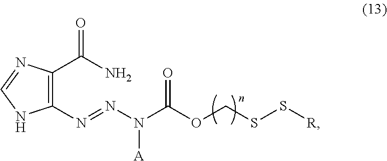
wherein A is selected from the group consisting of alkyl, haloalkyl, benzyl, halobenzyl, methyl, 2-chloroethyl, and ethyl methanesulfonate,
or a salt, solvate, enantiomer, diastereomer, geometric isomer or tautomer thereof.

15. The compound of claim 1, wherein Drug is a phosphoramide mustard.

16. The compound of claim 15, wherein the compound of formula (1) is: 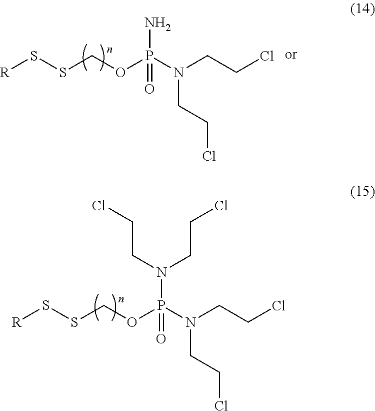
or a salt, solvate, enantiomer, diastereomer, geometric isomer or tautomer thereof.

17. The compound of claim 1, wherein the Drug is o-thioquinone methide.

18. The compound of claim 17, wherein the compound of formula (1) is: 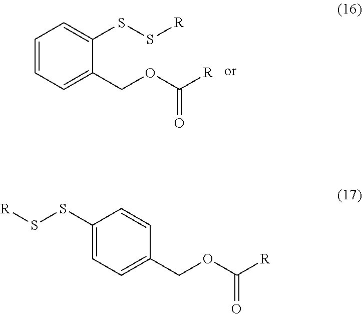
or a salt, solvate, enantiomer, diastereoisomer, geometric isomer or tautomer thereof.

19. A compound of formula (18): 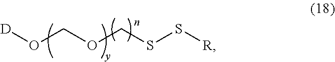
wherein:
D is a DNA binding or nuclear localizing moiety,
R is selected from the group consisting of a biomarker targeting moiety, a tumor targeting moiety, a DNA targeting moiety, C₁-C₆ alkyl, and aryl,
each occurrence of n is independently an integer ranging from 1 to 4,
y is an integer ranging from 0 to 20, wherein y is optionally 2;
or a salt, solvate, enantiomer, diastereoisomer, geometric isomer or tautomer thereof.

20. (canceled)

21. The compound of claim 1, wherein R is a weakly acidic group having a pKa between about 4.5 and about 7.5.

22. The compound of claim 21, wherein R is selected from the group consisting of: 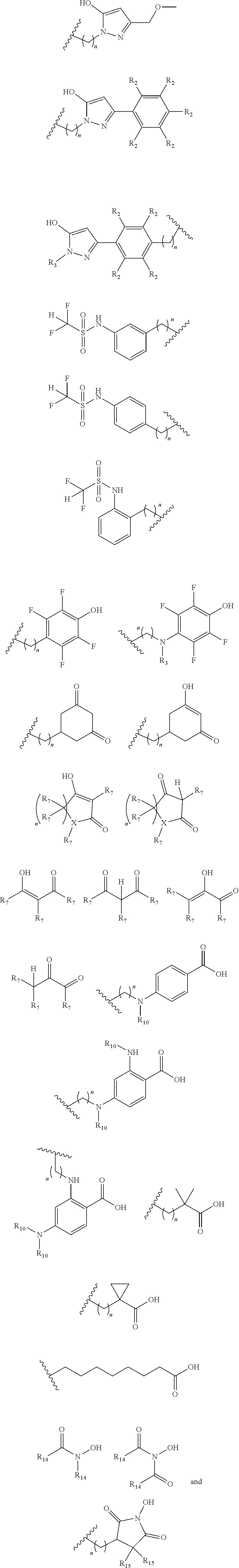
wherein each instance of R₂ is independently selected from the group consisting of H, F, Cl, hydroxy, methoxy, —NH₂, —NH-alkyl, —N(alkyl)₂, and alkyl;
R₃ is selected from the group consisting of H, methyl, ethyl, alkyl, phenyl, benzyl, haloaryl, —CH₂—O—CH₃, and —CH₂—CH₂—OH;
each instance of R₇ is independently selected from the group consisting of H, alkyl, phenyl, benzyl, an electron donating group, or a covalent bond to linker or drug;
X is CH, C-alkyl, or N;
at least one R₇ group comprises a covalent bond to Linker or Drug either directly or by displacing a hydrogen on alkyl, phenyl, benzyl or an electron donating group;
each instance of R₁₀ is independently selected from the group consisting of H, alkyl, or an electron donating group;
each instance of R₁₄ is independently an electron withdrawing group, an electron donating group or a covalent bond to Linker or Drug; and
at least one R₁₄ group comprises a covalent bond to Linker or Drug either directly or by displacing a hydrogen on an electron withdrawing or electron donating group;
each instance of R₁₅ is independently selected from the group consisting of: H, an electron withdrawing group or an electron donating group; and
n is an integer ranging from 0 to 4.

23. The compound of claim 21, wherein the compound is selected from the group consisting of:

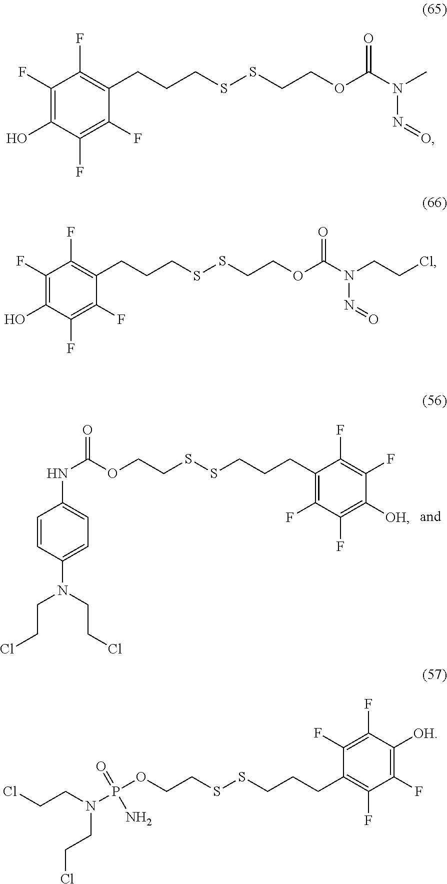 [SMILES: CN(N=O)C(=O)OCCSSCCCc1c(F)c(F)c(O)c(F)c1F ; O=NN(CCCl)C(=O)OCCSSCCCc1c(F)c(F)c(O)c(F)c1F ; O=C(Nc1ccc(N(CCCl)CCCl)cc1)OCCSSCCCc1c(F)c(F)c(O)c(F)c1F ; NP(=O)(OCCSSCCCc1c(F)c(F)c(O)c(F)c1F)N(CCCl)CCCl]

24-26. (canceled)

27. A method for treating a cancer in a subject in need thereof, the method comprising administering to the subject a therapeutically effective amount of the compound of claim 1.

28. (canceled)

29. The method of claim 27, wherein the cancer is at least one selected from the group consisting of melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-Hodgkins lymphoma, hepatocellular carcinoma, bladder cancer, and lung cancer.

30-33. (canceled)
